(19) United States
(12) Patent Application Publication
(10) Pub. No.: US 2014/0296894 A1
(43) Pub. Date: Oct. 2, 2014

(54) LIQUID EJECTION DEVICE AND MEDICAL APPARATUS

(71) Applicant: SEIKO EPSON CORPORATION, Tokyo (JP)

(72) Inventors: Hideki KOJIMA, Matsumoto-shi (JP); Hirokazu SEKINO, Chino-shi (JP); Takeshi SETO, Chofu-shi (JP)

(73) Assignee: SEIKO EPSON CORPORATION, Tokyo (JP)

(21) Appl. No.: 14/227,150

(22) Filed: Mar. 27, 2014

(30) Foreign Application Priority Data
Mar. 28, 2013 (JP) 2013-067734

(51) Int. Cl. A61B 17/3203

(52) U.S. Cl. CPC A61B 17/3203; A61B 2017/32032; A61B 2560/02; A61B 2218/007
USPC 606/167; 239/589

(57) ABSTRACT

A liquid ejection device ejecting a liquid includes: an ejection tube that has an ejection port ejecting the liquid; an actuator that communicates with the ejection tube and applies pulsation to the liquid that is flowed into and then supplies the liquid to the ejection tube; a casing that includes the ejection tube and accommodates the actuator on an inside thereof; a suction channel that is provided in the casing and is capable of sucking the liquid around the ejection port; and a suction adjustment mechanism that has an operation section for adjusting a degree of the suction of the suction channel, in which the operation section is provided in a position near the ejection port from the actuator of the casing.

This application claims the benefit of Japanese Patent Application No. 2013-67734, filed on Mar. 28, 2013. The content of the aforementioned application is incorporated herein by reference in its entirety.

### BACKGROUND

1. Technical Field

The present invention relates to technology of a liquid ejection device that ejects a liquid and a medical apparatus that uses the liquid ejection device.

2. Related Art

For example, technology regarding a liquid ejection device that ejects a liquid is disclosed in JP-A-2008-82202. JP-A-2008-82202 describes the liquid ejection device that converts a liquid to pulsed flow using an actuator having a piezoelectric element and ejects the liquid from an ejection port in a pulsed manner.

The liquid ejection device includes a casing that is grasped by a user. Various configuration members including the actuator are stored in the casing. Further, if other functions are added to the liquid ejection device in addition to the function for ejecting the liquid, it is necessary to further store a configuration member in the casing to realize the functions. In order for the user to grasp the casing, it is necessary to ensure an appropriate thinness thereof. So far, arrangement of the configuration members stored in the casing is not sufficiently devised considering the thinness (slim) of the casing. Further, the arrangement of the configuration members isnot sufficiently devised from in terms of how a load is applied or operability of the casing when the user grasps the casing.

### SUMMARY

An advantage of some aspects of the invention is to solve at least a part of the problems described above, and the invention can be implemented as the following aspects.

(1) An aspect of the invention provides a liquid ejection device. The liquid ejection device includes: an ejection tube that has an ejection port ejecting a liquid; an actuator that communicates with the ejection tube and applies pulsation to the liquid that is flowed into and then supplies the liquid to the ejection tube; a casing that includes the ejection tube and accommodates the actuator therein; a suction channel that is provided in the casing and is capable of sucking the liquid around the ejection port; and a suction adjustment mechanism that has an operation section for adjusting a degree of the suction of the suction channel. The operation section is provided in a position near the ejection port from the actuator of the casing. According to the liquid ejection device of this aspect, since the operation section is provided in the position near the ejection port from the actuator, it is possible for the suction adjustment mechanism that has the operation section and the actuator not to overlap each other in a particular direction inside the casing, and to ensure appropriate thinness of the casing. Further, it is possible for a user to be fully aware of a sensation of pressing of the operation section with a balance between gravity of the actuator having a high proportion of a load and a force that is applied to the operation section, thereby reducing a force that is additionally applied.

(2) In the liquid ejection device of the aspect described above, the suction adjustment mechanism may have a hole section communicating with the outside of the casing and the suction channel. According to this aspect, since the liquid ejection device has the hole section communicating with the outside of the casing and the suction channel, it is possible to adjust the degree of the suction by a pressure of the suction channel.

(3) In the liquid ejection device of the aspect described above, the degree of the suction may be gained as a size of a closed area on the hole section increases. According to this aspect, it is possible to adjust the degree of the suction by a relatively simple operation such as closing the surface area of the hole section.

(4) Another aspect of the invention provides a medical apparatus using the liquid ejection device described above. According to this aspect, it is possible to provide the medical apparatus having good usability.

Not all of a plurality of configuration elements included in each aspect of the invention described above is essential. A configuration element that is a part of the plurality of configuration elements described above may be appropriately changed, deleted and replaced with other, new configuration elements, and partial deletion of a limited content may be performed. Further, an independent aspect of the invention may be achieved by combining a part of or all of technical characteristics that are included in one aspect of the invention described above with a part of or all of technical characteristics that are included in another aspect of the invention described above.

For example, an aspect of the invention may be implemented as a device that includes one or more elements among five elements of the ejection tube, the actuator, the casing, the suction channel and the suction adjustment mechanism. That is, the device may have or may not have the ejection tube. Further, the device may have or may not have the actuator. Further, the device may have or may not have the casing. Further, the device may have or may not have the suction channel. Further, the device may have or may not have the suction adjustment mechanism. For example, the ejection tube may be configured as an ejection tube having an ejection port that ejects the liquid. For example, the actuator may be configured as an actuator that communicates with the ejection tube and applies a pulsation to the liquid flowed from the outside, and then supplies the liquid to the ejection tube. For example, the casing may be configured as a casing that includes the ejection tube in one end thereof and stores the actuator therein. For example, the suction channel may be configured as a suction channel that is provided in the casing and is capable of sucking the liquid around the ejection port. For example, the suction adjustment mechanism has the operation section for adjusting the degree of the suction of the suction channel. For example, such a device can be implemented as the liquid ejection device and can be also implemented as another device other than the liquid ejection device. According to the aspect described above, it is possible to solve at least one of various problems of small size, low cost, resource saving, ease of manufacturing, improvement of usability and the like. A part or all of the technical characteristics of each aspect of the liquid ejection device described above may be applied to the device.

The invention can be implemented in various aspects other than the device. For example, the invention can be implemented in an aspect of a method for ejecting the liquid or a method for manufacturing the liquid ejection device.

### BRIEF DESCRIPTION OF THE DRAWINGS

The invention will be described with reference to the accompanying drawings, wherein like numbers reference like elements.

FIG. 1 is an explanatory view describing a configuration of a liquid ejection device.

FIG. 2 is an explanatory view describing an internal structure of a handpiece.

FIG. 3 is an explanatory view schematically illustrating a force acting on the handpiece.

### DESCRIPTION OF EXEMPLARY EMBODIMENTS

### A. FIRST EMBODIMENT

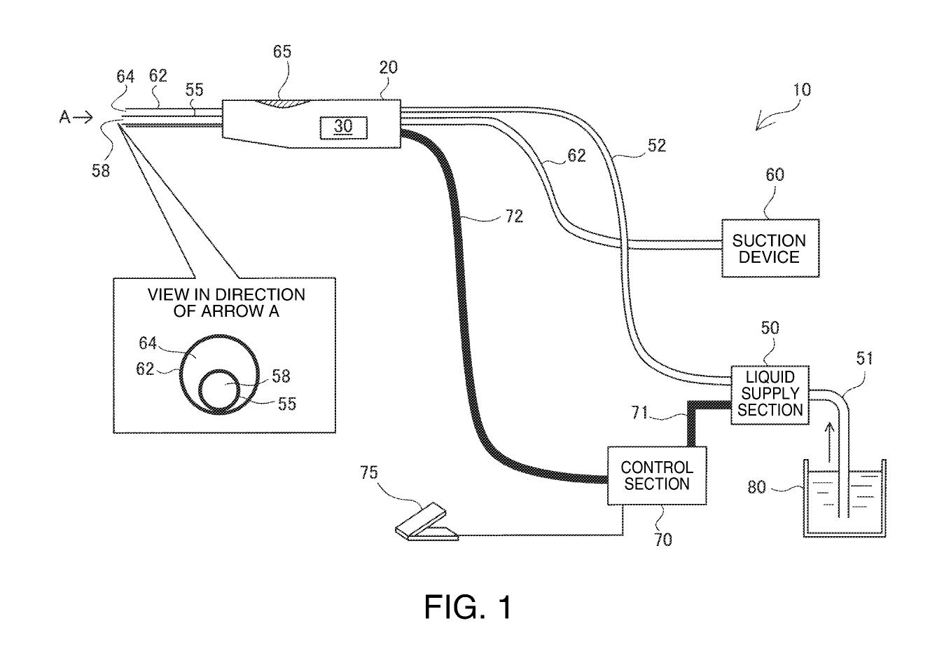

FIG. 1 is an explanatory view describing a configuration of a liquid ejection device 10 as a first embodiment of the invention. The liquid ejection device 10 of the embodiment is a medical apparatus used in medical institutions and has a function as a scalpel for performing incision on or excision of a diseased part by ejecting the liquid with respect to the diseased part or a medical apparatus that ejects a liquid medicine.

The liquid ejection device 10 includes a handpiece 20 as a casing, a liquid supply section 50, a suction device 60, a control section 70 and a liquid container 80. The liquid supply section 50 and the liquid container 80 are connected by a connection tube 51. The liquid supply section 50 and the handpiece 20 are connected by a liquid supply channel 52. In the embodiment, the connection tube 51 and the liquid supply channel 52 are formed by a resin.

The liquid container 80 accommodates physiological saline solution as the liquid. The liquid supply section 50 supplies the sucked liquid from the liquid container 80 through the connection tube 51 to the handpiece 20 through the liquid supply channel 52. Moreover, in the embodiment, the physiological saline solution is employed as the liquid, but the invention is not limited to the embodiment and it is possible to employ various types of liquids such as sterile water or pure water.

The handpiece 20 is a device that is grasped and operated by a user of the liquid ejection device 10. The handpiece 20 includes an actuator 30, an ejection tube 55, an ejection port 58, and a suction force adjustment mechanism 65. Pulsation having a predetermined frequency is applied to the liquid, which is supplied from the liquid supply section 50 to the handpiece 20 through the liquid supply channel 52, by the actuator 30, and the liquid is supplied to the ejection tube 55. The liquid supplied to the ejection tube 55 is ejected from the ejection port 58 as a pulsed liquid. The user performs incision on or excision of a diseased part by applying the pulsed liquid ejected from the ejection port 58 to the diseased part of the patient. In the embodiment, the ejection tube 55 is formed of stainless steel. However, the ejection tube 55 may be formed of a material having a predetermined rigidity or higher such as other metals, for example, brass and the like, or reinforced plastic. The suction force adjustment mechanism 65 is a mechanism capable of adjusting a degree of the suction of the suction device 60 by the user. In the embodiment, as the degree of the suction, a suction amount per unit time is employed.

The control section 70 transmits a drive signal to the actuator 30 through a signal cable 72 and controls the liquid supply section 50 through a control cable 71 thereby performing control of a flow amount of the liquid that is supplied to the actuator 30. A foot switch 75 that is operated by a foot of the user is connected to the control section 70. When the user turns ON the foot switch 75, the control section 70 controls the liquid supply section 50 and performs the supply of the liquid to the actuator 30, and transmits the drive signal to the actuator 30. Therefore, the pulsation is applied to the liquid that is supplied to the actuator 30 and the pulsed liquid is ejected from the ejection port 58. Moreover, the expression “the liquid is ejected in a pulsed manner” means that the liquid is ejected in a state where changes in the flow amount or a flow speed of the liquid that is ejected are accompanied. Intermittent ejection in which the liquid is ejected by repeating the ejection and stop of the liquid is included in the form in which the liquid is ejected in the pulsed manner. However, as long as the flow amount or the flow speed of the liquid changes, it is not necessary for the form to be the intermittent ejection.

The suction device 60 is a device that sucks the liquid, excised matter or the like (hereinafter, also referred to as suction matter) around the ejection port 58. The suction device 60 and the handpiece 20 are connected by a suction channel 62. The suction channel 62 extends near a leading end of the ejection tube 55 through inside the handpiece 20. The ejection tube 55 is inserted into the inside of the suction channel 62. As illustrated in a direction of arrow A in FIG. 1, a channel (hereinafter, also referred to as a gap channel) in which the liquid, which is sucked from a suction port 64 that is the leading end of the suction channel 62, flows, is formed between an outer wall of the ejection tube 55 and an inner wall of the suction channel 62. The liquid that flows from the suction port 64 into the gap channel is sucked into the suction device 60 through a suction channel 62. The suction amount per unit time that is sucked from the suction port 64 is capable of being adjusted by operating the suction force adjustment mechanism 65 by the user.

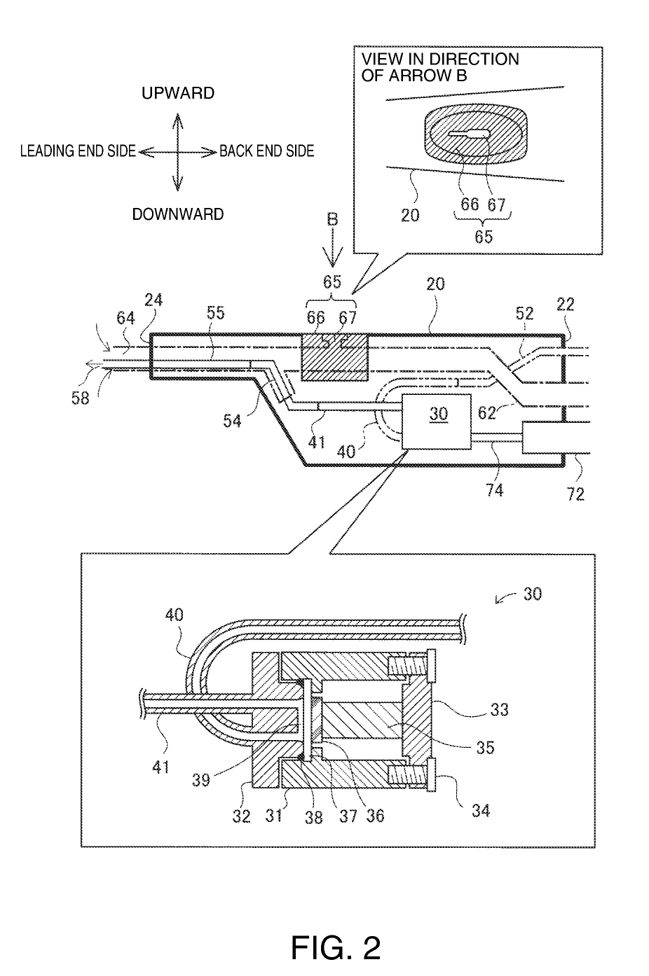

FIG. 2 is an explanatory view describing an internal structure of the handpiece 20. As described above, the actuator 30 is accommodated inside the handpiece 20. As illustrated in a lower section of the view, the actuator 30 includes a first case 31, a second case 32, a third case 33, a bolt 34, a piezoelectric element 35, a reinforced plate 36, a diaphragm 37, a gasket 38, an inlet channel 40, and an outlet channel 41. The first case 31 and the second case 32 are bonded by facing each other. The first case 31 is a cylindrical member. One end section of the first case 31 is closed by fixing the third case 33 with the bolts 34. The piezoelectric element 35 is disposed in a space that is formed inside the first case 31.

The piezoelectric element 35 is a multilayered piezoelectric element. One end section of the piezoelectric element 35 is fixed to the diaphragm 37 through the reinforced plate 36. The other end section of the piezoelectric element 35 is fixed to the third case 33. The diaphragm 37 is formed of a metal thin film and a peripheral section thereof is fixed to the first case 31. A liquid chamber 39 is formed between the diaphragm 37 and the second case 32. The volume of the liquid chamber 39 is changed by driving of the piezoelectric element 35.

The inlet channel 40 into which the liquid flows and the outlet channel 41 from which the liquid flows are formed in the second case 32. The inlet channel 40 extends from a position deviated from a center of an end surface of the second case 32 and is bent upward in a U-shape in the view, and stretches to a back end section 22 of the handpiece 20. As described above, it is possible to reduce a curvature of the U-shape portion of the inlet channel 40 by bending the inlet channel 40. Further, it is possible to reduce a combined width of the inlet channel 40 stretching to the back end section 22 and the actuator 30, and as a result, it is possible for the handpiece 20 that accommodates the actuator 30 and the inlet channel 40 to be thin. Further, it is possible to position the center of gravity of the handpiece 20 on the side of a leading end section 24 by stretching the inlet channel 40 from the side of the leading end section 24 of the actuator 30.

The liquid supply channel 52 is connected to the inlet channel 40. The ejection tube 55 is connected to the outlet channel 41 through a connection tube 54. The liquid supplied from the liquid supply section 50 is supplied to the liquid chamber 39 through the liquid supply channel 52. When the piezoelectric element 35 vibrates at a predetermined frequency, the volume of the liquid chamber 39 is changed through the diaphragm 37 and the liquid that is accommodated is pressurized. The liquid that is pressurized is discharged from the ejection port 58 through the outlet channel 41, the connection tube 54 and the ejection tube 55.

The suction channel 62 communicates throughout the leading end section 24 from the back end section 22 of the handpiece 20. On the inside of the handpiece 20, the connection tube 54 and the ejection tube 55 are inserted into the suction channel 62. The suction channel 62 is led out from the leading end section 24 of the handpiece 20 together with the ejection tube 55. The suction force that sucks the liquid or the like from the suction port 64 by the suction channel 62 is capable of being adjusted in the suction device 60 and is capable of being adjusted by operating an operation section 66 of the suction force adjustment mechanism 65 included in the handpiece 20 by the user.

The suction force adjustment mechanism 65 includes the operation section 66 and a hole section 67. The suction force adjustment mechanism 65 is a member that is formed of a resin. A channel forming a part of the suction channel 62 is formed inside the suction force adjustment mechanism 65. Practically, the suction channel 62 is connected to both ends of the channel formed in the suction force adjustment mechanism 65. The operation section 66 is a portion among the suction force adjustment mechanism 65 which is exposed to the outside of the handpiece 20 and is a portion that is operated with a finger of the user.

The hole section 67 communicates with the suction channel 62 and the operation section 66. As illustrated in a direction of arrow B of FIG. 2, an opening section of the hole section 67 is formed in the operation section 66. The user opens and closes the hole section 67 with a finger when grasping the handpiece 20. The suction force adjustment mechanism 65 adjusts the flow amount of air flowing from the outside into the suction channel 62 through the hole section 67 and adjusts the suction amount per unit time according to the size of the surface area of the hole section 67 that is closed by a user. If the suction force adjustment mechanism 65 is directed upward with respect to gravity, the suction matter is extremely difficult to be sucked from the hole section 67 to the outside and then the function thereof is preferably exerted. When grasping the handpiece 20, the user adjusts the suction amount per unit time of the suction device 60 by directing the suction force adjustment mechanism 65 in the upward direction and by pressing the hole section 67 included in the operation section 66 downward with a finger. Moreover, when grasping the suction force adjustment mechanism 65 in the upward direction with respect to the gravity, a position of each configuration of the liquid ejection device 10 is determined so that the function of the suction force adjustment mechanism 65 or operability of the user is suitable, but the suction force adjustment mechanism 65 does not force the user to necessarily use the handpiece 20 in the upward direction. Hereinafter, the description is given by defining that the direction in which the suction force adjustment mechanism 65 is provided in the handpiece 20 is an upward direction.

The signal cable 72 is inserted from the back end section 22 of the handpiece 20. Two electrode wires 74 consisting of plus and minus inserted into the signal cable 72 are connected to the piezoelectric element 35 inside the actuator 30. The drive signal transmitted from the control section 70 is transmitted to the piezoelectric element 35 through the electrode wires 74 inside the signal cable 72. The piezoelectric element 35 expands and contracts, based on the drive signal.

Here, a relative positional relationship between the operation section 66 included in the suction force adjustment mechanism 65 and the actuator 30 is described. The operation section 66 is disposed on the side of the leading end of the handpiece 20 from the actuator 30. Particularly, the operation section 66 including an opening section of the hole section 67 that is pressed with a finger of the user is disposed on the side of the leading end of the handpiece 20 from the actuator 30. It has the following effects by having such a positional relationship.

The suction force adjustment mechanism 65 and the actuator 30 have predetermined sizes inside the handpiece 20. Various configuration members such as the liquid supply channel 52, the inlet channel 40, the outlet channel 41, the connection tube 54, the ejection tube 55, and the suction channel 62 are accommodated inside the handpiece 20. These configuration members are laid substantially parallel to the long axial direction of the handpiece 20. Therefore, the sizes of these configuration members affect the size of the diameter of a cross section perpendicular to the long axial direction of the handpiece 20 that has a columnar shape. In the embodiment, it is at least possible for the suction force adjustment mechanism 65 and the actuator 30 to avoid being overlapped in the direction perpendicular to the long axial direction of the handpiece 20 by disposing the suction force adjustment mechanism 65 (the operation section 66) on the side of the leading end from the actuator 30. Therefore, it is possible to avoid increase in the diameter of the cross section perpendicular to the long axial direction of the handpiece 20. As a result, for the handpiece 20, it is possible to ensure an appropriate thinness that is easy to be grasped by the user and to realize slimming.

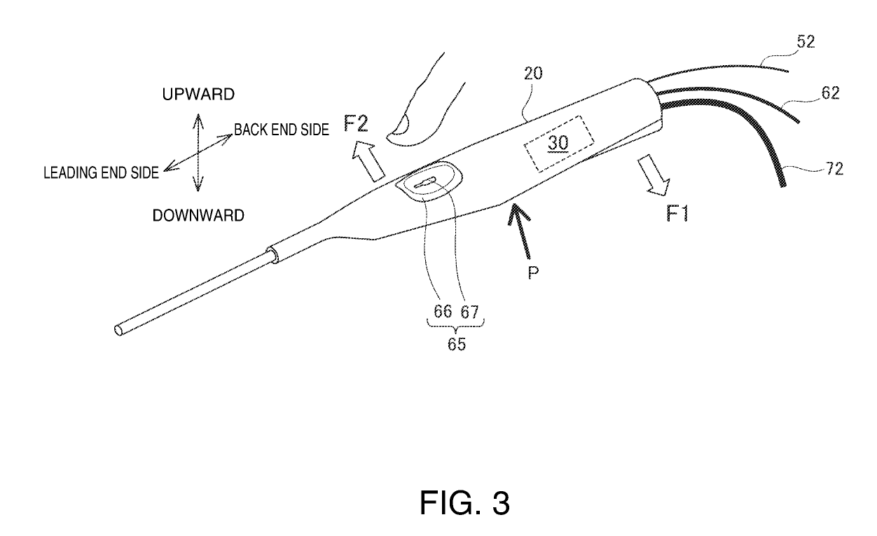

FIG. 3 is an explanatory view schematically illustrating a force acting on the handpiece 20 when the user grasps the handpiece 20. Since most of materials configuring the actuator 30 are configured of a metal member, a proportion of a load of the actuator 30 among a load of the handpiece 20 as a whole is high. Therefore, a gravity of the handpiece 20 is positioned near the actuator 30. As described in FIG. 3, when the user grasps the handpiece 20 on about a grasping position P between the actuator 30 and the operation section 66 as a fulcrum, the load of the actuator 30 or the loads of the liquid supply channel 52, the suction channel 62 and the signal cable 72 which are disposed in the back end of the handpiece 20 acts as a downward force F1 with respect to the handpiece 20. An upward force F2 (a rotational force) acts on the operation section 66 that is positioned on the side of the leading end of the handpiece 20 about the grasping position P as the fulcrum due to the force F1.

Therefore, when pressing the hole section 67 of the operation section 66, the user can be fully aware of a sensation of pressing the hole section 67. Further, the upward force F2 acting on the operation section 66 acts as a part of a force that is necessary to press the hole section 67 by the user. Thus, it is possible to reduce the force that is additionally applied to press the hole section 67 by the user. As a result, it is possible to reduce fatigue of the user when grasping the handpiece 20 for a long time.

### B. MODIFICATION EXAMPLE

Moreover, the invention is not limited to the embodiment or embodiments described above and it is possible to implement the invention in various aspects within the scope that does not depart from the spirit thereof and, for example, the following variations are possible.

### B1 Modification Example 1

In the embodiment described above, the effects are described as a case where the grasping position P is positioned between the actuator 30 and the operation section 66, but if the grasping position P is positioned on the side of the back end from the operation section 66, it is possible to obtain effects similar to those of the above description. For example, if the grasping position P is positioned near the actuator 30, weights of the liquid supply channel 52, the suction channel 62 and the signal cable 72 act as the downward force F1 with the grasping position P as the fulcrum. Therefore, the upward force F2 (the rotational force) acts on the operation section 66 that is positioned on the side of the leading end of the handpiece 20 with the grasping position P as the fulcrum. Therefore, the user can be fully aware of the sensation of pressing the hole section 67 of the operation section 66. The upward force F2 acting on the operation section 66 acts as a part of a force that is necessary for the user to press the hole section 67 by the action of the force F1. Thus, it is possible to reduce the force that is additionally applied by the user to press the hole section 67. As a result, it is possible to reduce fatigue of the user when grasping the handpiece 20 for a long time.

### B2 Modification Example 2

In the embodiment described above, the suction force adjustment mechanism 65 has the configuration having the hole section 67, but the invention is not limited to the embodiment and it is possible to employ various aspects. For example, the suction force adjustment mechanism 65 has a pressing mechanism that presses the suction channel 62 and the suction amount per unit time can be adjusted by narrowing or widening a cross-sectional area of the channel of the suction channel 62. In addition, a controller that electrically adjusts the degree of the suction of the suction device 60 may be included in the handpiece 20 as the suction force adjustment mechanism 65. Then, an operation signal is transmitted to the suction device 60 by operating operation buttons, a dial, a lever or the like included in the operation section 66 by the user, and the suction device 60 changes the degree of the suction depending on the operation signal. Also in this case, it is possible to obtain the same advantage as that of the embodiment described above.

### B3 Modification Example 3

In the embodiment described above, the liquid ejection device 10 is used as the medical apparatus. However, the liquid ejection device 10 maybe used as an apparatus other than the medical apparatus. For example, the liquid ejection device 10 may be used as a cleaning apparatus for removing soil of an object by applying the liquid that is ejected to the object, or as a depiction apparatus that draws characters, graphics or the like by the liquid that is ejected. Also in this case, it is possible to obtain the same advantage as that of the embodiment described above.

### B4 Modification Example 4

In the embodiment described above, the physiological saline solution is employed as the liquid, but the invention is not limited to the embodiment and it is possible to employ various types of liquids such as sterile water or ultrapure water.

### B5 Modification Example 5

In the embodiment described above, the suction amount per unit time is used as the degree of the suction that is adjusted by the suction force adjustment mechanism, but the invention is not limited to the embodiment and, for example, various types of physical quantities capable of defining the degree of the suction such as using the suction force are employed and the physical quantities may be adjusted.

### B6 Modification Example 6

In the embodiment described above, the aspect is employed in which the ejection tube 55 is inserted into the suction channel 62 as a disposition relationship of the ejection tube 55 and the suction channel 62 which are led out from the leading end section 24 of the handpiece 20, but the invention is not limited to the embodiment. For example, it is possible to employ various forms such as employing an aspect that the outer wall of the ejection tube 55 and the outer wall of the suction channel 62 are led out from the leading end section 24 by being adjacent to each other. In order to suck the liquid around the ejection port 58 from the suction port 64, it is preferable that the ejection tube 55 and the suction channel 62 be integrally led out from the leading end section 24 as in the embodiment described above and in the Modification Example.

What is claimed is:

1. A liquid ejection device ejecting a liquid, comprising:
an ejection tube that has an ejection port ejecting the liquid;
an actuator that communicates with the ejection tube and applies pulsation to the liquid that is flowed into and then supplies the liquid to the ejection tube;
a casing that includes the ejection tube and accommodates the actuator therein;
a suction channel that is provided in the casing and is capable of sucking the liquid around the ejection port; and
a suction adjustment mechanism that has an operation section for adjusting a degree of the suction of the suction channel,
wherein the operation section is provided in a position near the ejection port from the actuator of the casing.

2. The liquid ejection device according to claim 1,
wherein the suction adjustment mechanism has a hole section communicating with the outside of the casing and the suction channel.

3. The liquid ejection device according to claim 2,
wherein the degree of the suction is gained as a size of a closed area on the hole section increases.

4. A medical apparatus using the liquid ejection device according to claim 1.

5. A medical apparatus using the liquid ejection device according to claim 2.

6. A medical apparatus using the liquid ejection device according to claim 3.
